(19) United States
(12) Patent Application Publication
(10) Pub. No.: US 2012/0164230 A1
(43) Pub. Date: Jun. 28, 2012

(54) Soluble Nanoparticles as Delivery Systems for Prodrugs

(76) Inventors: Rodney Feazell, Sacramento, CA (US); Nozomi Nakayama-Ratchford, Mountain View, CA (US); Hongjie Dai, Cupertino, CA (US); Stephen J. Lippard, Cambridge, MA (US)

(21) Appl. No.: 12/115,709

(22) Filed: May 6, 2008

Related U.S. Application Data
(60) Provisional application No. 60/916,683, filed on May 8, 2007.

(51) Int. Cl. A61K 31/282; C08B 37/02; C07K 5/09; C07K 16/00; C12N 5/071; C07F 15/00; A61K 9/18; B82Y 5/00

(52) U.S. Cl. 424/490; 556/137; 536/51; 514/492; 530/331; 530/391.7; 435/375; 977/773; 977/746; 977/906

(57) ABSTRACT

Compounds and methods are disclosed in which a prodrug can be delivered in an elevated oxidative state to cells by means of graphitic nanoparticles to which the prodrug is attached by a hydrophilic polymer and which have been made soluble by a hydrophilic polymer, such as PEG. The graphitic nanoparticle may be a single walled carbon nanotube (SWNT). The prodrug may be a DNA-binding metal-based drug. Exemplified is a platinum(IV) complex c,c,t-[Pt(NH₃)₂Cl₂(OEt)(O₂CCH₂CH₂CO₂H)], which is nearly nontoxic to testicular cancer cells, but displays a significantly enhanced cytotoxicity profile when attached to the surface of amine-functionalized soluble SWNTs. An amine functionality on the hydrophilic polymer may be used to link the prodrug.

### CROSS-REFERENCE TO RELATED APPLICATIONS

This application claims priority from U.S. Provisional Patent Application No. 60/916,683 filed on May 8, 2007, which is hereby incorporated by reference in its entirety.

### STATEMENT OF GOVERNMENTAL SUPPORT

This invention was made with U.S. Government support under National Institutes of Health Grant Number CA34992 awarded by the National Cancer Institute. The U.S. Government has certain rights in this invention.

### l REFERENCE TO SEQUENCE LISTING, COMPUTER PROGRAM, OR COMPACT DISK

None

### BACKGROUND OF THE INVENTION

1. Field of the Invention

The present invention relates to the field of nanoparticles such as carbon nanotubes, and to the field of delivery of molecules to cells.

2. Related Art

Presented below is background information on certain aspects of the present invention as they may relate to technical features referred to in the detailed description, but not necessarily described in detail. The discussion below should not be construed as an admission as to the relevance of the information to the claimed invention or the prior art effect of the material described.

Attempts to devise platinum drugs that surpass the anticancer properties of cisplatin (cis-[Pt(NH₃)₂Cl₂]) have produced many compounds that display biological activity, but only a handful of these have shown any real promise in clinical trials.¹ This loss of activity in the body can be associated with poor circulation and delivery to the tumor as well as deactivation mechanisms that irreversibly alter the chemistry of these molecules, particularly those of platinum(II), rendering them ineffective.² We can circumvent many pathways that deactivate platinum(II) drug candidates by utilizing substitutionally more inert platinum(IV) compounds as prodrugs or by using carrier molecules as delivery systems.^{1,3} We recently demonstrated the utility of this approach by attaching cell-sensitizing estradiol units to platinum(IV) compounds which, upon entry into the cell, were reduced to release the cytotoxic compound cis-[Pt(NH₃)₂Cl₂].⁴

Another delivery system that has emerged as a highly effective means of transporting certain molecular cargos across the cell membrane is the functionalized soluble single-walled carbon nanotube (SWNT), which carries molecules into cells through clathrin-dependent endocytosis.⁵ SWNTs tethered to substrates by disulfide linkages use the reducing environment of endosomes into which they are taken to selectively release their cargo only following cellular internalization.⁶

### Specific Patents and Publications

Dai et al. US 2006/0275371, “Hydrophobic Nanotubes and Nanoparticles as Transporters for the Delivery of Drugs into Cells,” published Dec. 7, 2006, describes the coupling of PEG-PL (polyethylene glycol-phospholipid) and its use for conjugation to DNA and proteins and for noncovalent binding to SWNTs (single walled nanotubes) in Examples 17-26.

Kam et al. “Carbon nanotubes as multifunctional biological transporters and near-infrared agents for selective cancer cell destruction,” Proc. Nat. Acad. Sci., 102 (33) 11600-11605 (2005) discloses that selective cancer cell destruction can be achieved by functionalization of an SWNT with a folate moiety, selective internalization of SWNTs inside cells labeled with folate receptor tumor markers, and NIR-triggered cell death, without harming receptor-free normal cells.

Feazell et al. “Soluble single-walled carbon nanotubes as longboat delivery systems for platinum(IV) anticancer drug design,” J. Am. Chem. Soc., 129:8438-8439 (Published on web 6/15/2007) discloses SWNT delivery of platinum (IV) compounds as taught herein. U.S. Pat. No. 5,871,710 to Bogdanov, et al., issued Feb. 16, 1999, entitled “Graft co-polymer adducts of platinum (II) compounds,” discloses a biocompatible graft co-polymer adduct including a polymeric carrier, a protective chain linked to the polymeric carrier, a reporter group linked to the carrier or to the carrier and the protective chain, and a reversibly linked Pt(II) compound.

U.S. Pat. No. 7,138,520 to Lippard, et al., issued Nov. 21, 2006, entitled “Coordination complexes having tethered therapeutic agents and/or targeting moieties, and methods of making and using the same,” discloses coordination complexes that have a covalently attached therapeutic agent and/or covalently attached targeting moiety. Upon release of the therapeutic agent or targeting moiety from a metal ion of the coordination complex, the resulting coordination complex is intended to be therapeutically effective. Upon release of the therapeutic agent the therapeutic agent is intended to be therapeutically effective as well.

### BRIEF SUMMARY OF THE INVENTION

The following brief summary is not intended to include all features and aspects of the present invention, nor does it imply that the invention must include all features and aspects discussed in this summary.

The present invention provides a nanoparticle complex for delivery of a prodrug to a cell, where the prodrug is converted to an active drug by chemical action within the cellular environment. The nanoparticle complex comprises three components: a hydrophobic nanoparticle; a hydrophobic polymer noncovalently bound to the nanoparticle; and a prodrug attached to the surface of the nanoparticle through a cleavable linkage to the hydrophilic polymer. The prodrug is converted to an active drug within an endosome.

The present invention further provides a nanoparticle complex for delivery of an active agent into a cell. The nanoparticle complex comprises a nanoparticle having an extended aromatic structure, an amphiphilic polymer for bonding to the nanoparticle and solubilizing it, and a prodrug coupled to the hydrophilic polymer.

The present invention also provides a method for preparing a nanoparticle complex for delivery of a prodrug of a reduced active agent inside a cell. According to this method, a nanoparticle having an extended aromatic surface is obtained in dispersed form. A hydrophilic polymer is then attached noncovalently to the nanoparticle, preferably through an aliphatic portion, such as in a phospholipid, whereby the aliphatic portion is hydrophobically bound to the nanoparticle, and is linked to the hydrophilic polymer. Next, a prodrug is linked to the hydrophilic polymer. Finally, a stable aqueous suspension of the complex is formed.

In addition, the present invention provides a method for delivering an effective amount of a prodrug for conversion of the prodrug to an active agent inside a cell. The method comprises administering the prodrug in a complex by contacting the cell with the complex for a time sufficient to allow internalization of the complex. According to this method, the complex comprises a nanoparticle having a graphite surface, a hydrophilic polymer bound to the nanoparticle, and a prodrug linked to the hydrophilic polymer through a cleavable linkage.

In certain aspects, the present invention relates to delivery of a prodrug attached through a cleavable linker and having a prodrug, which is converted into an active pH, where both cleavage and conversion happen as a result of delivery of the prodrug to a relatively acidic environment, such as an endosome.

In certain other aspects, the present invention relates to the preparation of a metallic prodrug, which is linked to a carrier by an axial linker, where the carrier is a hydrophilic polymer having a functional terminus, such as amine terminated PEG.

### BRIEF DESCRIPTION OF THE DRAWINGS

FIG. 1A shows the structure of a compound 2, c,c,t-[Pt(NH₃)₂Cl₂(OEt) (O₂CCH₂CH₂CO₂H)]; FIG. 1B shows an SWNT-tethered conjugate, SWNT-Pt(IV); and FIG. 1C shows a phospholipid tethered amine having a PEG chain between the amine and the phospholipid used for anchoring the complex to the nanotube. The “ . . . ” in FIG. 1C indicates the linkage to the Pt compound, i.e., the formula shown in FIG. 1B.

FIG. 2 is a graph showing the cytotoxicity of free compound 2 and SWNT-tethered compound 2 in NTera-2 cells. The cytotoxicity of the free platinum(IV) complex increased by greater than 100-fold when attached to the surface of the functionalized SWNTs.

FIG. 3 is a photograph showing the fluorescence of a fluorophore co-tethered SWNT-Pt(IV), SWNT-Pt(IV)-F1, in NTera-2 cells. The nucleus was stained blue with a Hoechst (H33258) nuclear stain and the endosomes and portions of the cytoplasm were stained green (colors not shown).

FIG. 4 is a diagram illustrating the proposed cellular entry mechanisms of the SWNT-tethered complexes. The SWNTs were taken into testicular cancer cells by endocytosis where the drop in pH facilitates the reductive release of the platinum (II) core complex, which then readily diffuses throughout the cell, as determined by platinum atomic absorption spectroscopy. The entrapment of the SWNTs within the endosomes was confirmed by fluorescence microscopy of SWNTs containing both tethered fluorescein and platinum units. Fluorescence of SWNT-Pt(IV)-F1 in NTera-2 cells included a blue dye which was Hoechst (H33258) nuclear stain.

FIG. 5 is a graph of cyclic voltammograms of c,c,t-[Pt(NH₃)₂Cl₂(OEt) (O₂CCH₂CH₂CO₂H)] taken at pH 6 with varied scan rates (S3).

FIG. 6 is a graph of cyclic voltammograms of c,c,t-[Pt(NH₃)₂Cl₂(OEt)(O₂CCH₂CH₂CO₂H)] taken at pH 7.4 with varied scan rates (S7).

FIG. 7 is a graph showing cytotoxicity of cis-[Pt(NH₃)₂Cl₂ ] in NTera-2 cells incubated with and without the same amine-functionalized SWNT to which was tethered the platinum (IV)compound c,c,t-[Pt(NH₃)₂Cl₂(OEt)(O₂CCH₂CH₂CO₂H)]. This experiment demonstrated that the SWNT-NH₂ construct does not affect the cytotoxicity of cisplatin.

FIG. 8 is a graph showing cytotoxicity of cis-[Pt(NH₃)₂Cl₂] in NTera-2 cells compared to that of c,c,t-[Pt(NH₃)₂Cl₂(OEt)(O₂CCH₂CH₂CO₂H)] and unplatinated SWNT-NH₂. The concentration of SWNT-NH₂ is given in terms of total amine concentration.

FIG. 9 is a graph showing cytotoxicity of c,c,t-[Pt(NH₃)₂Cl₂(OH)(OEt)] (compound 1) in NTera-2 cells.

### DETAILED DESCRIPTION OF THE PREFERRED EMBODIMENT

### Definitions

Unless defined otherwise, all technical and scientific terms used herein have the same meaning as commonly understood by those of ordinary skill in the art to which this invention belongs. Although any methods and materials similar or equivalent to those described herein can be used in the practice or testing of the present invention, the preferred methods and materials are described. Generally, nomenclatures utilized in connection with, and techniques of, cell and molecular biology and chemistry are those well known and commonly used in the art. Certain experimental techniques, not specifically defined, are generally performed according to conventional methods well known in the art and as described in various general and more specific references that are cited and discussed throughout the present specification. For purposes of the clarity, following terms are defined below.

The term “prodrug” means any compound that when administered to a biological system generates a biologically active compound as a result of spontaneous chemical reaction(s), enzyme catalyzed chemical reaction(s), and/or metabolic chemical reaction(s), or a combination of each. Prodrugs must undergo some form of a chemical transformation to produce the compound that is biologically active or is a precursor of the biologically active compound. In some cases, the prodrug is biologically active, usually less than the drug itself, and serves to improve drug efficacy or safety through improved oral bioavailability, pharmacodynamic half-life, etc. The biologically active compounds include, for example, anticancer agents, radiation potentiating agents, as well as other agents such as antihypertensive agents, antiviral agents, and antibiotic agents. In a preferred embodiment, the present prodrugs are activated in a lower than physiological pH (7.4), i.e., about pH 6, that exists in an endosome. The term “prodrug” is not used to refer to active agents that are inactive solely by virtue of their coupling to a second entity (such as a nanoparticle). In other words, prodrugs of the present invention must be activated in some way beyond mere cleavage of an active drug from another molecular entity. The term “metal prodrug” means a prodrug wherein the metal is an active agent (“active metal prodrug”), such as metal which binds to DNA (e.g., platinum), or a prodrug where the metal is used to complex an active agent. In either case, the metal is reduced to activate the prodrug.

The prodrug is preferably a “small molecule,” which is a chemical or other moiety, other than a polypeptide or nucleic acid, which can act to affect biological processes. Small molecules can include any number of therapeutic agents presently known and used, or can be small molecules synthesized in a library of such molecules for the purpose of screening for biological function(s). Small molecules are distinguished from macromolecules by size. The small molecules of this invention have molecular weight less than about 5,000 daltons (Da), preferably less than about 2,500 Da, more preferably less than 1,000 Da, most preferably less than about 500 Da.

The term “aliphatic,” as used herein, includes both saturated and unsaturated, straight chain (i.e., unbranched), branched, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl,” “alkynyl” and the like. Furthermore, as used herein, the terms “alkyl,” “alkenyl,” “alkynyl” and the like encompass both substituted and unsubstituted groups (i.e., are alkoxy, thioalkyl, or alkyl amino groups). In certain embodiments, as used herein, “lower alkyl” is used to indicate those alkyl groups (cyclic, acyclic, substituted, unsubstituted, branched or unbranched) having 1-6 carbon atoms.

The aliphatic (lipid) alkyl groups employed in the lipids of the invention preferably contain 4-20, more preferably 10-20 aliphatic carbon atoms. In certain other embodiments, the lower alkyl, (including alkenyl, and alkynyl) groups employed in the invention contain 1-10 aliphatic carbon atoms. Illustrative aliphatic groups thus include, but are not limited to, methyl, ethyl, n-propyl, isopropyl, cyclopropyl, —CH₂-Cyclopropyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, hexyl, sec-hexyl, cyclohexyl, —CH₂-cyclohexyl moieties and the like, which again, may bear one or more substituents. Alkenyl groups include, but are not limited to, ethenyl, propenyl, butenyl, 1-methyl-2-buten-1-yl, and the like. Representative alkynyl groups include, but are not limited to, ethynyl, 2-propynyl(propargyl), 1-propynyl and the like. The aliphatic groups are hydrophobic and adsorb to the hydrophobic nanoparticle.

The term “alkoxy” (or “alkyloxy”), or “thioalkyl” as used herein refers to an alkyl group, as previously defined, attached to the parent molecular moiety through an oxygen atom or through a sulfur atom. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkoxy include, but are not limited to, methoxy, ethoxy, propoxy, isopropoxy, n-butoxy, tert-butoxy, neopentoxy and n-hexoxy. Examples of thioalkyl include, but are not limited to, methylthio, ethylthio, propylthio, isopropylthio, n-butylthio, and the like.

The term “alkylamino” refers to a group having the structure —NHR wherein R is alkyl, as defined herein. The term “dialkylamino” refers to a group having the structure —N(R)₂, wherein R is alkyl, as defined herein. The term “aminoalkyl” refers to a group having the structure NH₂ R—, wherein R is alkyl, as defined herein. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkylamino include, but are not limited to, methylamino, ethylamino, isopropylamino and the like.

The term “carbon nanotube” means a tube that contains a sheet of graphene rolled into a cylinder as small as 1 nm in diameter. Both single-walled nanotubes (SWNTs) and multiwalled nanotubes (MWNTs), with many concentric shells, have been synthesized. The electronic properties of a nanotube depend on the angle (chirality) with which it is rolled up—the present nanotubes can act as metallic conductors, small-gap semiconductors, or large-gap semiconductors. Carbon nanotubes may include other materials. For example, some carbon nanotubes are “metallic” in that they conduct electricity. Metallic tubes have shown ballistic conduction on length scales of a micron or more. The present carbon nanotubes may be metallic or semiconducting. Nanotubes are also the stiffest known material, with a Young's modulus of ˜1 TPa. The nanotubes herein may include structures that are not entirely carbon, such as BCN nanotubes. They may also be graphene in other forms. This includes a single sheet of graphene formed into a sphere, which constitutes a carbon nanosphere, commonly referred to as Buckminsterfullerene, a buckyball or simply fullerene.

The expression “dosage unit form” means a physically discrete unit of therapeutic agent appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed as the prodrug; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts (see, for example, Goodman and Gilman's, “The Pharmacological Basis of Therapeutics,” Tenth Edition, A. Gilman, J. Hardman and L. Limbird, eds., McGraw-Hill Press, 155-173, 2001, which is incorporated herein by reference in its entirety).

The term “effective amount” means a sufficient amount of agent to cause a detectable decrease in the condition to be modulated in the cell, or an increase in the desired effect, e.g., cell death, as measured by any of the assays described in the examples herein. The exact amount required will vary from subject to subject, depending on the species, age, and general condition of the subject, the severity of the proliferative disease or other disease, the particular therapeutic agent, its mode of administration, and the like.

The term “endosome” means a membranous organelle to which molecules internalized by a cell via endocytosis are transferred. The endosomal apparatus within polarized cells can be subdivided into two types of compartments: a “housekeeping endosome” that recycles certain internalized cell surface components (e.g., transferrin receptor and low-density lipoprotein receptor) primarily to the basolateral surface; and a “specialized endosome” from which vesicles targeted to the apical surface leave. These are not typically considered to be discrete compartments; that is, components may flow in both directions between these compartments. Proton pumps within the endosome permit the endosome interior to reach a pH of between 5 and 6.

The term “hydrophilic polymer” means a material that has the property of dissolving in, absorbing, or mixing easily with water, and comprises repeating units constituting an MW of at least 200 Da up to 8,000 Da or more. The preferred hydrophilic polymer is PEG, as defined below. Other exemplary materials for this purpose include poly(hydroxyalkyl methacrylates); poly(N-vinyl-2-pyrrolidone); anionic and cationic hydrogels; polyelectrolyte complexes; poly(vinyl alcohol) having a low acetate residual and cross-linked with glyoxal, formaldehyde, or glutaraldehyde; methylcellulose cross-linked with a dialdehyde, a mixture of agar and sodium carboxymethyl cellulose, a water-insoluble, water-swellable copolymer produced by forming a dispersion of finely divided copolymer of maleic anhydride with styrene, ethylene, propylene, butylene or isobutylene cross-linked with from about 0.001 to about 0.5 mole of a polyunsaturated cross-linking agent per mole of maleic anhydride in the copolymer as disclosed in U.S. Pat. No. 3,989,586, water-swellable polymers of N-vinyl lactams as disclosed in U.S. Pat. No. 3,992,562, and the like (See U.S. Pat. No. 4,207,893 to Michaels, issued Jun. 17, 1980, entitled “Device using hydrophilic polymer for delivering drug to biological environment.”) Another exemplary polymer is dextran, which may be branched. The dextran straight chain consists of α1→6 glycosidic linkages between glucose molecules, while branches begin from α1→3 linkages (and in some cases, α1→2 and α1→4 linkages as well). One may apply Dextran 10, Dextran 40 and Dextran 70; MW=10,000, 40,000 and 70,000 Da, respectively) at a concentration analogous to those described for PEG.

Hydrophilic polymers suitable for use herein include polyethylene glycol (PEG), polyoxyethylene, polymethylene glycol, polytrimethylene glycols, polyvinyl-pyrrolidones, and derivatives thereof with PEG being particularly preferred. The polymers can be linear or multiply branched, and will not be substantially crosslinked. Other suitable polymers include polyoxyethylenepolyoxypropylene block polymers and copolymers. Polyoxyethylene-polyoxypropylene block polymers having an ethylene diamine nucleus (and thus having four ends) are also available and may be used in the practice of the invention.

The hydrophilic polymer used here will render the nanoparticles soluble when attached thereto in sufficient numbers. A precise hydrophobic/hydrophilic measurement can be made as described in Bowe et al., “Design of compounds that increase the absorption of polar molecules,” Proc. Natl. Acad. Sci. USA, Vol. 94, pp. 12218-12223, October 1997.

The hydrophilic polymer may be branched, e.g., having 4 branches, thus providing three attachment sites for the prodrug active agent. For example, 2, 3, 4 and 8 arm branched PEGs are available from NOF Corporation, Tokyo Japan. Further description of multi-arm hydrophilic molecules is found in “Multi-arm block copolymers as drug delivery vehicles,” U.S. Pat. No. 6,730,334.

The term “nanoparticle” means a material having the properties of a carbon nanotube insofar as the material is essentially hydrophobic, inert and atomically smooth. The present nanoparticles will typically have a diameter on the order of the diameter of an SWNT or MWNT (preferably 10-20 nm, not more than 100 nm) or smaller, and length not more than about 20 μm, preferably of not more than 50-500 nm in length. They will be atomically ordered and generally chemically inert, such as a nanowire (see, e.g., “Controlled growth of highly uniform, axial/radial direction-defined, individually addressable InP nanowire arrays,” Premila Mohan et al 2005 Nanotechnology 16 2903-2907, and US PGPUB 20050221083 to Belcher, et al., published Oct. 6, 2005, entitled “Inorganic nanowires,” hereby incorporated by reference), fullerenes, fullerenols, etc. The term “nanoparticle” is also intended to include nanostructured materials <100-1000 nm in at least one of the three dimensions such as tubes, wires, particles and crystals. The term nanoparticle also includes carbon black, whose primary particles range in size from 10 nm to 500 nm. Carbon blacks are commercially available in a variety of particle sizes and morphologies. The term “nanoparticle” also includes hydrophobic polymeric particles, such as spheres of “nanoparticle” size. i.e., less than 1000 nm, e.g., polystyrene beads of 20, 50 or 100nm as exemplified below.

The term “hydrophobic polymer” is used herein to mean any polymer resistant to wetting, or not readily wet, by water (i.e., having a lack of affinity for water). A hydrophobic polymer typically will have a surface free energy of about 40 dynes/cm (10⁻⁵ Newtons/cm or N/cm) or less.

Examples of hydrophobic polymers which can be used to form nanoparticles include, by way of illustration only, polylactide, polylactic acid., polyolefins, such as poylethylene, poly(isobutene), poly(isoprene), poly(4-methyl-l-pentene), polypropylene, ethylene-propylene copolymers, and ethylenepropylene-hexadiene copolymers; ethylene-vinyl acetate copolymers; styrene polymers, such as poly(styrene), poly(2-methylstyrene), styrene-acrylonitrile copolymers having less than about 20 mole-percent acrylonitrile, and styrene-2,2,3,3,-tetrafluoro-propyl methacrylate copolymers. Further examples are given in U.S. Pat. No. 6,673,447, hereby incorporated by reference.

As exemplified by polystyrene and ordered graphene carbon sheets, tubes, spheres, or other shapes, the preferred nanoparticle has an “extended aromatic structure” (i.e., materials which comprise at least one repeating unit that includes an extended aromatic ring or a polycyclic aromatic ring system containing 2, 3, 4 or more rings, preferably at least two of the rings being fused, from about 3 to 8 ring members in each ring). The term “extended” aromatic ring system refers to an aromatic group, particularly a single ring group such as phenyl, that is either fused to another ring or contains one or more unsaturated ring substituents such as cyano, alkenyl, alkynyl, alkanoyl, nitro, etc. Compounds with extended aromatic structures are further exemplified in U.S. Pat. No. 3,197,475 issued Jul. 27, 1965. One form of elemental carbon, graphite, consists of an extended aromatic-ring structure where the carbon atoms are bound by sp² -bonds and delocalized pi-bonds. As the number of rings increase, these pi-bonding electron orbitals become degenerate across the entire structure and end up existing at a level with little or no gap between the valence bands and conduction bands.

Another exemplary nanoparticle having an extended aromatic structure (i.e., a graphene surface) is a coated metal or metal oxide nanocrystal. Seo et al., ‘FeCo/graphitic-shell nanocrystals as advanced magnetic resonance imaging and near-infrared agents,” Nature Materials 5, 971-976 (2006) describes, in the preferred embodiment, the preparation of a scalable chemical vapor deposition method to synthesize FeCo/single-graphitic-shell nanocrystals that are soluble and stable in water solutions. In addition, U.S. Pat. No. 6,843,919 to Klabunde, et al., issued Jan. 18, 2005, entitled “Carbon-coated metal oxide nanoparticles,” discloses methods of preparation of nanoparticles having from about 10-20% by mass carbon coating layer, based upon the total weight of the final coated composite taken as 100% by mass. The coating layer is graphitic and carbonaceous in nature and will comprise at least about 90% by mass carbon and preferably at least about 98% by mass carbon. Hollow graphitic nanoparticles may also be prepared, as described in US PGPUB 2006/0198949 by Phillips; Jonathan et al., published Sep. 7, 2006, entitled “Preparation of graphitic articles.” Sutter et al., “Assembly and interaction of Au/C core-shell nanoparticles,” Surface Science Volume 600, Issue 18, Pages 3525-4404 (15 Sep. 2006) discloses that, at high temperatures (400-800° C.), Au particles are transformed into Au/C core-shell structures via encapsulation into curved, fullerene-like C shells, thus describing another method for preparing nanoparticles having a graphitic surface.

The term “organic amphiphilic molecule” means an amphiphile containing a hydrophobic portion, such as an alkyl group of at least 3 carbon atoms linked to a hydrophilic portion, e.g., a “hydrophilic polymer”, for stabilizing the molecule in aqueous solution. The alkyl group may be a lipid attached to a polar head group, which itself is hydrophilic or is bonded to a hydrophilic polymer. The hydrophilic polymer is preferably a polymer such as PEG.

The term “PEG” means Polyethylene glycol, a polymer with the structure (—CH₂CH₂O—)ₙ that is synthesized normally by ring opening polymerization of ethylene oxide. The PEG used herein will impart water (and thus serum) solubility to the hydrophobic nanoparticle and lipid portion of the polar lipid. The polymer is usually linear at molecular weights (MWs)≦10 kD. The PEG used here will have an MW below 5,400, preferably below 2,000, or about 45 repeating ethylene oxide units. However, the higher MW PEGs (higher “n” repeating units) may have some degree of branching. Polyethylene glycols of different MWs have already been used in pharmaceutical products for different reasons (e.g., increase in solubility of drugs). Therefore, from the regulatory standpoint, they are very attractive for further development as drug or protein carriers. The PEG used here should be attached to the nanoparticles at a density adjusted for the PEG length. For example, with PL-PEG 2000, we have an estimate of ˜4 nm spacing between PEG chains along the tube. At this spacing, PEG 5400 is too long and starts to block interaction with cell surface. For PEG at ˜1 nm distance, the PEG MW should be less than about 200, to allow hydrophobicity.

For coupling proteins to PEG, usually monomethoxy PEG [CH₃(OCH₂CH₂)ₙOH] is first activated by means of cyanuric chloride, 1,1′-carbonyldiimidazole, phenylchloroformate, or succidinimidyl active ester before the addition of the prodrug. In most cases, the activating agent acts as a linker between PEG and the prodrug, and several PEG molecules may be attached to one nanoparticle. The pharmacokinetics and pharmacodynamics of the present nanotubes-PEG-prodrug conjugates are expected to be somewhat dependent on the MW of the PEG used for conjugation. Generally the presently used PEG will have a molecular weight of approximately 100-2,000 Daltons. Further description of chemistries for linking the prodrug to the PEG may be found in U.S. Pat. No. 6,566,406, which also describes the use of succinic anhydride.

The present PEG may also be a modified PEG such as PolyPEG® (Warwick Effect Polymers, Ltd., Coventry, England). PolyPEGs® are a new range of materials suitable for the attachment of polyethylene glycol (PEG) to therapeutic proteins or small molecules. These are prepared using Warwick Effect Polymers' polymerization technology, (See U.S. Pat. No. 6,310,149) and contain terminal groups suitable for conjugation with functionalized prodrugs.

The term “polar lipid” refers to a molecule having an aliphatic carbon chain with a terminal polar group. Preferred polar lipids include but are not limited to acyl carnitine, acylated carnitine, sphingosine, ceramide, phosphatidyl choline, phosphatidyl glycerol, phosphatidyl ethanolamine, phosphatidyl inositol, phosphatidyl serine, cardiolipin and phosphatidic acid. Further polar lipids are exemplified in U.S. Pat. No. 6,339,060, “Conjugate of biologically active compound and polar lipid conjugated to a microparticle for biological targeting,” to Yatvin, et al., hereby incorporated by reference.

The term “phospholipid” means a molecule having an aliphatic carbon chain with a terminal phosphate group. Typically the phospholipids will be comprised of a glycerol backbone, attached to two fatty acid (aliphatic groups) esters and an alkyl phosphate. Suitable phospholipids for use in this invention include, but are not limited to, dimyristoyl phosphatidylcholine, distearoyl phosphatidylcholine, dilinoleoyl-phosphatidylcholine (DLL-PC), dipalmitoyl-phosphatidylcholine (DPPC), soy phophatidylchloine (Soy-PC or PCs) and egg phosphatidycholine (Egg-PC or PCE). Suitable phospholipids also include, but are not limited to, dipalmitoyl phosphatidylcholine, phosphatidyl choline, or a mixture thereof. Exemplified below are 1,2-dipalmitoyl-sn-glycero-3 phosphoethanolamine phospholipid and 1,2-Distearoyl-sn-Glycero-3-Phosphoethanolamine.

The term “stable” means a solution or suspension in a fluid phase wherein solid components (i.e., nanotubes and prodrugs) possess stability against aggregation sufficient to allow manufacture and delivery to a cell and which maintain the integrity of the compound for a sufficient period of time to be detected and preferably for a sufficient period of time to be useful for the purposes detailed herein. The present complexes are “dispersed” in that they are soluble or form stable suspensions. They may contain some degree of nanoparticle aggregation, but are sufficiently individualized such that they may readily be formed in such preparations, as opposed to continuous sheets or clumps of such complexes.

The term “soluble” refers to solubility in water or aqueous medium, including physiological fluids, which contain dissolved inorganic salts and other components. It is not intended to require that 100% of the “soluble” nanoparticles be in solution, or that the particles be in a true solution, or that they remain in solution for a lengthy period of time. It is required that they remain in stable suspension, without settling or clumping, preferably for at least about 30 days.

The term “substituted” whether preceded by the term “optionally” or not, and substituents contained in formulas of this invention, refer to the replacement of hydrogen atoms in a given structure with a specified substituent molecule. When more than one position in any given structure may be substituted with more than one substituent selected from a specified group, the substituent may be either the same or different at every position. As used herein, the term “substituted” is contemplated to include all permissible substituents of organic compounds.

In a broad aspect, the permissible substituents include acyclic and cyclic, branched and unbranched, carbocyclic and heterocyclic, aromatic and non-aromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of cancer.

The term “targeting agent” means a molecule, which is a specific ligand for a cell surface protein, and can be coupled to the present nanoparticle complex without rendering the complex insoluble or preventing release of the attached drug. The targeting agent is preferably linked to the hydrophilic polymer, e.g., through an amine terminus on a PEG molecule. The exemplified targeting agent is a cyclic RGD peptide, as described, for example, in U.S. Pat. No. 5,192,746 to Lobl, et al., issued Mar. 9, 1993, entitled “Cyclic cell adhesion modulation compounds,” and in “Cyclic RGD Peptide-Labeled Liposomes for Targeting Drug Therapy of Hepatic Fibrosis in Rats,” J Pharmacol Exp Ther. 2007 May 17 (E_PUB).

The present targeting agents may also include antibodies and antibody fragments, such as Affibody® molecules, which are 58-amino acid three-helix bundle proteins directed to different targets by combinatorial engineering of staphylococcal protein A.

The term “antibody” includes antibodies obtained from both polyclonal and monoclonal preparations, as well as: hybrid (chimeric) antibody molecules (see, for example, Winter et al. (1991) Nature 349:293 299; and U.S. Pat. No. 4,816,567); F(ab′)2 and F(ab) fragments; Fv molecules (noncovalent heterodimers, see, for example, Inbar et al. (1972) Proc Natl Acad Sci USA 69:2659 2662; and Ehrlich et al. (1980) Biochem 19:4091 4096); single-chain Fv molecules (sFv) (see, for example, Huston et al. (1988) Proc Natl Acad Sci USA 85:5879 5883); dimeric and trimeric antibody fragment constructs; minibodies (see, e.g., Pack et al. (1992) Biochem 31:1579 1584; Cumber et al. (1992) J Immunology 149B: 120 126); humanized antibody molecules (see, for example, Riechmann et al. (1988) Nature 332:323 327; Verhoeyan et al. (1988) Science 239:1534 1536; and U.K. Patent Publication No. GB 2,276,169, published 21 Sep. 1994); and any functional fragments obtained from such molecules, wherein such fragments retain specific-binding properties of the parent antibody molecule. Exemplary targeting antibodies include antibodies to cellular receptors such as the HER-2 or EGF receptor, as well as antibodies to cell surface cluster determinant (“CD”) antigens. Other antibody targets include alpha fetal protein, CA-125, HLA and other antigens.

### Overview

The present disclosure concerns methods and compositions for the delivery of prodrugs to cells. The prodrugs are exemplified by platinum compounds that are active in the +2 oxidative state but may be prepared as prodrugs in the +4 oxidative state (platinum(IV)). By conjugating such prodrugs to the present delivery systems, the prodrugs are taken into the reducing environment of the cell's endosomes, where the active molecule is released upon intracellular reduction. Exemplified below is the construction of a SWNT-tethered platinum(IV) conjugate that effectively delivers a lethal dose of cis-[Pt(NH₃)₂Cl₂] upon reduction inside the cell. The present methods involve combining the ability of platinum (IV) complexes to resist ligand substitution, and hence deactivation of the active drug form, with the capacity of SWNTs to act as a lipophilic carrier for shuttling smaller molecules across cell membranes.

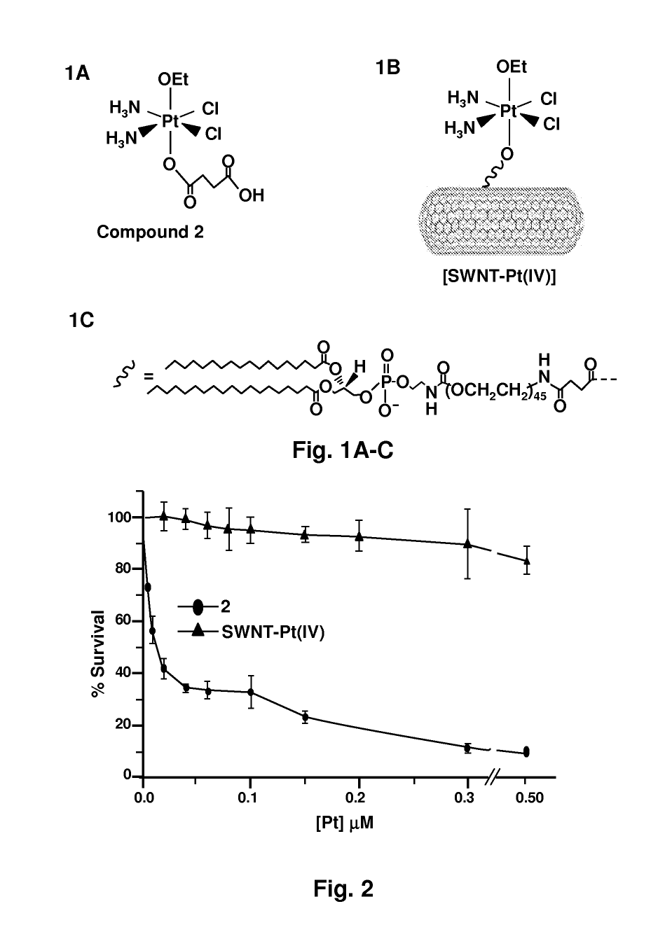

As shown in FIG. 1a, Compound 2, c,c,t-[Pt(NH₃)₂Cl₂(OEt)(O₂CCH₂CH₂CO₂H)], a platinum(IV) construct, was synthesized so as to be capable of being tethered through one of its axial ligands to an amine-functionalized PEG which is noncovalently linked to the SWNT surface. Compound 2 was chosen as the prodrug cargo in order to allow us to regenerate and release the toxic molecule cis-[Pt(NH₃)₂Cl₂] upon intracellular reduction. The precursor complex, c,c,t-[Pt(NH₃)₂Cl₂(OH)(OEt)], compound 1, was synthesized in a manner similar to other trans-alkoxohydroxoplatinum(IV) complexes by the oxidation of cis-[Pt(NH₃)₂Cl₂] in dilute ethanolic hydrogen peroxide.⁷

The axial hydroxide of compound 1 is sufficiently nucleophilic to attack succinic anhydride upon mild heating to afford the asymmetrically substituted trans-alkoxocarboxylato complex 2. The term “axial” refers, as is known in chemical terminology, to distinguish “equatorial” groups as illustrated in this case in FIG. 1 as the four groups comprising Cl₂ and H₃N.

The SWNTs used in this study were functionalized by nonspecific binding of lipid portions of phospholipid tethered amines to the nanotube surface.^{5a} A polyethyleneglycol chain between the amine and the anchoring phospholipid serves to solubilize the SWNTs and extend the functional group away from the nanotube surface. Treating c,c,t-[Pt(NH₃)₂Cl₂(OEt)(O₂CCH₂CH₂CO₂H)] (compound 2) with 1-ethyl-3-[3-dimethylaminopropyl]carbodiimide hydrochloride (EDC) and N-hydroxysuccinimide (NHS) creates the N-succinimidyl ester, which readily forms amide linkages with the PEG-tethered primary amines on the SWNT surface. After the tethering reaction, dialysis through a 3500 MW cutoff membrane separates any free platinum from the much larger SWNT-Pt(IV) conjugates. The result is a SWNT “longboat” (carrier) that carries a cargo average of 65 platinum (IV) centers per nanotube (range of 60-100) as determined by atomic absorption spectroscopy (AAS). Formation of the amide bond is not expected to significantly alter the reduction potential of the platinum(IV) center since the electronic interaction of the two positions is insulated by two methylene groups.

In order to evaluate the feasibility of this method for delivering a toxic dose of platinum, cytotoxicity assays of SWNT-Pt(IV) were conducted using the testicular carcinoma cell line NTera-2. Example 3 below describes these experiments, in which the MTT assay was used to determine cell viability following treatment for four days with either the free platinum(IV) complex 2 or with the SWNT-tethered analog (FIG. 2). Compared to cis-[Pt(NH₃)₂Cl₂] (IC₅₀=0.05 μM) the cytotoxicity of the asymmetrically substituted platinum(IV) compound 2 is insignificant (IC₅₀>>0.5 mM). The SWNT-Pt(IV) conjugate, however, shows a substantial increase in toxic character with respect to the free complex 2 (IC₅₀=0.02) and surpasses that of cis-[Pt(NH₃)₂Cl₂]. Without wishing to be bound to any particular theory, it is thought that this increase is due to the two-fold increase in uptake of Pt relative to free cisplatin.

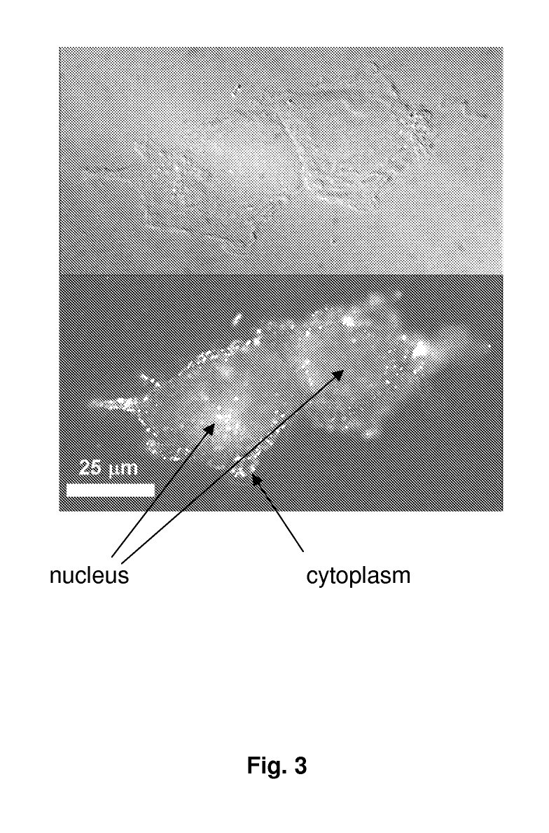

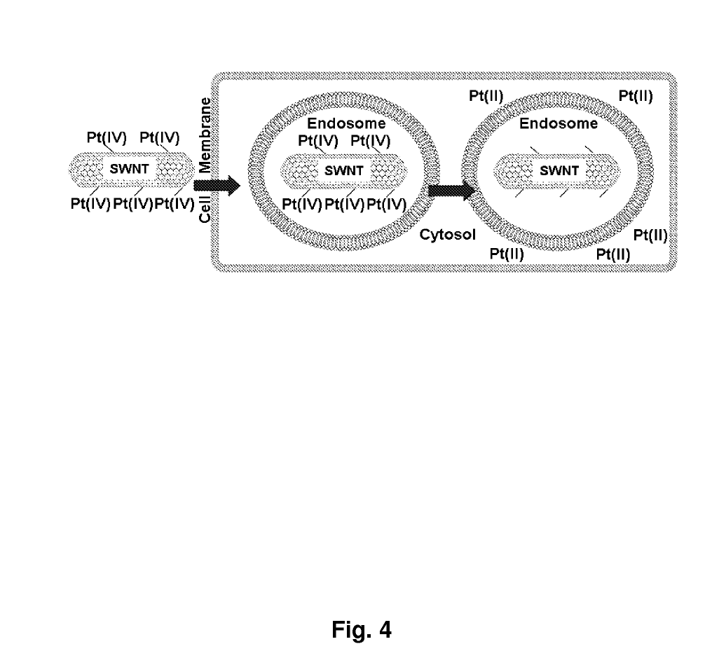

In order to investigate the mechanism by which the SWNTs transport Pt(IV) compounds into cells and release them once inside we devised a strategy to track their intracellular location. To follow the movement of the SWNTs we cotethered a fluorescein-based fluorophore(6-carboxy-2′,7′-dichlorofluorescein-3′,6′-diacetatesuccinimidyl ester) to our SWNT-Pt(IV) conjugates.⁸ After a 1.5 h incubation with the SWNT-Pt(IV)-F1 conjugates the now fluorescent SWNTs were readily located within small (˜2 μm) vesicles in the cell (FIG. 3). This finding supports previous reports that SWNTs are taken up by cells through endocytosis.^{5c, 5f} To determine the locale of platinum once inside the cells and to learn whether or not it was released from the SWNT delivery vessel, extracts were taken from the cytosol and nucleus of cells incubated with SWNT-Pt(IV). AAS analysis of both fractions showed considerable amounts of platinum (23 ng Pt/mg protein in cytosol and 36 ng Pt/mg protein in nucleus) in cells that were incubated for 3 h with platinum concentrations of 1.0 μM. These levels were 6-8 times higher than the platinum concentration found in cells incubated with the untethered complex 2 and also twice the platinum concentration than measured in cells incubated with cis-[Pt(NH₃)₂Cl₂]. This intracellular accumulation and distribution of platinum demonstrates that functionalized SWNTs are an effective tool for transport and delivering small molecule platinum prodrugs.

Exemplified below are materials and methods in which the platinum(IV) complex c,c,t-[Pt(NH₃)₂Cl₂(OEt)(O₂CCH₂CH₂CO₂H)], which is nearly nontoxic to testicular cancer cells, displays a significantly enhanced cytotoxicity profile when attached to the surface of amine-functionalized soluble SWNTs. The soluble nanotubes internalized the platinum(IV) compound through endocytosis, providing six times the concentration that the untethered complex is able to achieve on its own. Once confined within the endosomes, the lower pH environment facilitates release of platinum as its core compound, cis-[Pt(NH₃)₂Cl₂], by reduction and concomitant loss of the axial ligands by which it is tethered to the SWNT surface. Atomic absorption spectroscopy confirms the distribution of platinum throughout the cell interior, while fluorescence microscopy verifies the trapped nature of the SWNTs. These results demonstrate the value of SWNTs for transporting platinum(IV) prodrugs across cell membranes where they can be reductively released as active platinum(II) species. The tethering and endosomal release of platinum(IV) prodrugs can be generally applied to any platinum(IV) compound that has a functional group on its axial ligands capable of being coupled to one of the many types of functionalized SWNTs. By linking additional groups, such as cancer-cell targeting moieties, to the platinated SWNTs as long boat passengers it may be possible to achieve highly selective constructs for use in clinical trials. It is generally preferred that a number of hydrophilic polymers (e.g., 10-500) (typically 50-100) be attached to the nanoparticle, sufficient to render the particle essentially soluble. Similarly, there will be 10-500 prodrug molecular complexes per nanoparticle.

In addition to the embodiments contemplated by the examples below, one may also, given the present teachings, prepare other prodrugs, which are coupled to solubilized hydrophobic nanoparticles for delivery to cells. An example of a case where metal is used to incorporate an active agent in the complex is described in Parker et al., “A Novel Design Strategy for Stable Metal Complexes of Nitrogen Mustards as Bioreductive Prodrugs,” J. Med. Chem., 47 (23), 5683 -5689, 2004. In this embodiment, one uses polyamine mustards for conversion into hypoxia-selective prodrugs via complexation with metals. Other alternative embodiments may use dinuclear Pt, other forms of platinum drugs, such as carboplatin, or metals other than platinum. With regard to DNA-binding metal drugs, one may employ in the present complexes non-platinum prodrugs based on metal complexes such as palladium, ruthenium, rhodium, copper, and lanthanum. For example, one Ru(III) compound, [ImH][trans-Cl₄(Me₂SO)(Im)Ru(III)] (Im=imidazole, NAMI-A) successfully entered phase I clinical trials.

Non-metal prodrugs may also be employed in the present compositions. For example, Swift et al., “Activation of Adriamycin by the pH-dependent Formaldehyde-releasing Prodrug Hexamethylenetetramine,” Molecular Cancer Therapeutics, Vol. 2, 189-198, (February 2002), describes the use of hexamethylenetetramine (HMTA), which is known to hydrolyze under cellular conditions, to release six molecules of formaldehyde in a pH-dependent manner. This clinical agent has potential as a formaldehyde-releasing prodrug for the activation of Adriamycin. U.S. Pat. No. 6,030,997 to Eilat, et al., issued Feb. 29, 2000, entitled “Acid labile prodrugs,” describes further prodrugs which may be coupled as described herein.

The hypoxic environment of cancer cells is conducive to reduction of a prodrug into its active form, as illustrated below. Bioreductively activated drugs are widely in demand for their specific and selective targeting of diseased tissues. Nitroimidazoles have been used as prodrugs for many years. The use of nitroimidazoles in anti-tumor therapy stems from the differences observed in the environment of, and the physiological concentration of molecular oxygen in, tumor versus normal tissues. Tumor cells reside in a much more acidic environment than normal cells and, as discussed previously, are severely more hypoxic than normal cells. In the context of tumor therapy and diagnosis of hypoxia, 2-, 4-, or 5-nitroimidazoles may be used here as reduction activated prodrugs. See e.g., Shimamura, et al. Brit. J. Cancer (2003) 88: 307-13; Kasai et al., Bioorg. Med. Chem., (2001) 9: 453-64; Papadopoulou et al., Bioorg. Med Chem., (2004) 14: 1523-25. In particular, 2-nitroimidazoles have significantly higher reduction potentials (about −418 mV) as compared to unsubstituted nitrobenzene (about −486 mV) and hence, are known to be selective to hypoxic regions of tumors. The 2-nitroimidazoles, therefore, have been identified as one of the three structurally different classes of bioreactive drugs with selective toxicity towards hypoxic cells, the mitomycins and benzotriazine dioxides being the other two classes.

The 2-nitroimidazoles, mitomycins and benzotriazine dioxides, as bioreactive drugs, are activated selectively in the absence of oxygen to reactive intermediates that can damage deoxyribonucleic acid (DNA). In the case of 2-nitroimidazoles, several reactive reductive intermediates, namely, the nitroso and hydroxylamine intermediates, have been isolated and their interaction with DNA and cellular thiols have been assessed at the molecular level. See e.g., Cowan, D. S. M., et al., Br. J. Cancer, (1994)70: 1067-74; Brezden, C. B., et al., Biochem. Pharmacol, (1994) 48: 361-70. For reviews on bioreductive therapy and synthesis of nitroimidazole prodrugs, see e.g., Jaffar et al., Adv. Drug Del. Rev., (2001) 53: 217-28; Naylor, M. A. and P. Thomson, Mini Rev. Med. Chem., (2001) 1: 17-29; Hay et al., J. Med. Chem., (2003) 46: 5533-45.

Another type of prodrug that may be used in the present system is one that is activated by reduction of a disulfide prodrug into an active agent having a sulfhydryl group. See e.g., Fournie-Zaluski et al., “Brain renin-angiotensin system blockade by systemically active aminopeptidase A inhibitors: A potential treatment of salt-dependent hypertension,” Proc. Nat. Acad. Sci., 101(20) 7775-7780 (May 18, 2004). Because the thiol group of the prodrug (in that case RB 150) is engaged in a disulfide bridge, it is unable to interact with the zinc atom present in the active site of the target enzyme (there APA). However, in vivo the disulfide bridge of the prodrug can be cleaved by brain reductases, generating the active drug. The reducing environment of an endosome or hypoxic cell may also be used to cleave a disulfide linkage.

Another type of pH sensitive prodrug is described in Reincke et al., “Development of β-Lapachone Prodrugs for Therapy Against Human Cancer Cells with Elevated NAD(P)H :Quinone Oxidoreductase 1 Levels,” Clinical Cancer Research, Vol. 11, 3055-3064, Apr. 15, 2005. As reported there, β-Lapachone (3,4-dihydro-2,2-dimethyl-2H-naphtho[1,2-b]pyran-5,6-dione) has demonstrated significant antitumor activity against sarcoma 180 cells in vitro. The biologically inactive mono(arylimino) derivatives of β-lapachone seem to act as Schiffs bases and are converted to β-lapachone through a spontaneous hydrolytic reaction. The rates of hydrolysis of β-lapachone derivatives varied as a function of the strength of the electron-withdrawing substituent groups in the para position of the mono(arylimino) leaving group as well as the pH of the solution.

It is further contemplated that the complexes may include targeting agents, such as antibodies, for directing the prodrug-laden nanoparticles to selected cells for entry into the cells and release of their cargo inside the cell. The targeting molecules may also be directed to organelles. Antibodies are known to be useful for targeting cancerous cells. Other examples of targeting agents include the 14 amino acid peptide bombesin, as described for example in Ma et al., “In Vitro and In Vivo Evaluation of Alexa Fluor 680-Bombesin[7-14]NH(2) Peptide Conjugate, a High-Affinity Fluorescent Probe with High Selectivity for the Gastrin-Releasing Peptide Receptor,” Mol Imaging. 2007 July-September; 6(3):171-80. The 12 amino acid peptide FROP1 was identified by phage screening, as other targeting agents useful here may be. See, Zitzmann et al., “Identification and Evaluation of a New Tumor Cell-Binding Peptide, FROP-1, ” J Nucl Med. 2007 June; 48(6):965-972. Epub 2007 May 15. Other targeting agents that recognize particular cell types, e.g., VEGF, which binds to VEGF receptors, or folic acid, which binds to folate receptors, may be used. Analogs of folic acid with these same properties are known and may also be used. Analogs are discussed in Roos et al. “Toxicity of Folic Acid Analogs in Cultured Human Cells: A Microtiter Assay for the Analysis of Drug Competition,” PNAS, Jul. 15, 1987, vol. 84, no. 14, 4860-4864. Certain tumor cells overexpress the folic acid receptor.

The targeting agent may be coupled to the terminal of the hydrophilic polymer (e.g., coupled to an amine-terminated PEG) or the targeting agent may be coupled to the prodrug, e.g., the OEt cap in compound 2 may be replaced by a linking group such as a second succinate and coupled to a targeting molecule.

### EXAMPLES

### Example 1

### Preparation of Platinum (IV) Compounds, and Tethering to Amine-PEG-Phospholipid SWNT

Materials. cis-[Pt(NH₃)₂Cl₂]¹ and 6-carboxy-2′,7′-[dichlorofluorescein-3′,6′-[diacetate succinimidyl ester² were synthesized as previously described. Distilled water was purified by passage though a Millipore Milli-Q Biocel water purification system (18.2 MΩ) with a 0.22 μm filter. NHS, EDC, paraformaldehyde, and succinic anhydride were purchased from Aldrich. All other solvents and reagents were obtained from VWR International and used as received. ¹H NMR (Nuclear Magnetic Resonance) spectra were recorded on a Varian Mercury Inova-500 spectrometer and ¹⁹⁵Pt NMR spectra were recorded on a Varian Inova-500 spectrometer in the Massachusetts Institute of Technology Department of Chemistry Instrumentation Facility (MIT DCIF). Atomic absorption spectroscopic measurements were taken on a Perkin Elmer AAnalyst300 spectrometer.

Synthesis of SWNT-PL-PEG-NH₂. SWNTs made by a high pressure CO (Hipco) method were purchased and used to construct amine-functionalized SWNTs via non-specific interactions between the SWNT surface and a phospholipid.³⁻⁵ Raw Hipco SWNTs were sonicated in PL-PEG-NH₂ for one h followed by harsh centrifugation (2.4×10⁴ g, 6 h) to remove catalysts and large aggregates with individual tubes and small bundles left in the supernatant. Excess free PL-PEG-NH₂ was removed from the supernatant by filtration through 100kDa Millipore filters. The resulting solution contained SWNTs with an average length of about 200 nm as determined by atomic force microscopy. The molar concentration of SWNTs, measured as previously reported, was on the order of 400 nM.⁶ Between 50-100 amine groups were estimated to be present on each nanotube.⁴

Synthesis of c,c,t-[Pt(NH₃)₂Cl₂(OH)(OEt)], compound 1. cis-[Pt(NH₃) Cl₂] (0.200 g, 0.67 mmol) was suspended in 250 mL of absolute ethanol and heated to 70° C. To this suspension was added 0.5 mL of a 50% H₂O₂ solution with vigorous stifling. After 5 h at elevated temperature the solid dissolved to afford a bright yellow solution. The solution volume was reduced to near dryness on a rotary evaporator and 50 mL of ether was added to precipitate the product as a light yellow solid. The solid was collected and washed with ice cold ethanol and ether. Yield was 98% (0.236 g, 0.65 mmol). d⁶ DMF, ¹⁹⁵Pt NMR: δ=873.9 ppm. ¹H NMR: δ=10.44 s, 1H(OH); 6.018 s, br, 6H(NH₃); 3.58 q, 2H(CH₂); 1.10 q, 3H(CH₃) ppm. Melting point (d), 172-175° C.

Synthesis of c,c,t-[Pt(NH₃)₂Cl₂(OEt)(O₂CCH₂CH₂CO₂H)], compound 2. Compound 1 (0.050 g, 0.14 mmol) was dissolved in 2 mL of DMF (Dimethylformamide). To this solution was added succinic anhydride (0.021 g, 0.21 mmol) in 1 mL of DMF and the solution was stirred for four h at 75° C. The resulting solution was dried in vacuo to leave a dark yellow oil, which was dissolved in a small amount of acetone. Addition of ether precipitated a solid that was collected and dried in vacuo to leave the product as a pale yellow powder in 35% yield (0.023 g, 0.05 mmol). Diffraction quality crystals were gown by vapor diffusion of ether into a DMF solution of the compound. NMR (d⁶ DMF) ¹⁹⁵Pt: δ=920.2 ppm ¹H: δ=12.36 m, 1H(CO₂H); 6.14 m, 6H(NH₃); 3.52 s, 2H(CH₂); 2.95 s, 2H(CH₂); 2.78 s, 2H(CH₂); 1.01 t, 3H(CH₃) ppm. ESI-MS (−): Calcd=461.1 for [M−H]⁻, Found=460.8. Anal. Calcd for C₆H₁₆C₁₂N₂O₅Pt.H₂O: C, 15.01; H=3.78; N=5.83. Found: C, 14.72; H, 353; N, 6.28. Melting point(decomposition), 120-124° C. Crystallographic data for compound 2 were collected on a yellow block crystal with dimensions 0.15×0.10×0.10 mm. Measurements were made at 110 K on a Bruker SMART Apex CCD diffractometer using graphite-monochromated Mo kα radiation (λ=0.71073 Å). The structure was solved by direct methods after correction of the data for absorption using SADABS (Siemens Area Detector ABSorportion correction program). Crystallographic results are presented in supplemental materials to the above cited J. Am. Chem. Soc. paper by the present inventors (J. Am. Chem. Soc., 129:8438, 2007). There are two crystallographically independent molecules of 2 in the asymmetric unit with the terminal —CH₃ group of one of the molecules being disordered over two positions with an occupancy ratio of 35:65. There are two molecules of DMSO (dimethyl sulfoxide) and one water molecule per asymmetric unit. All non-hydrogen atoms were refined anisotropically and hydrogen atoms were placed in calculated positions.

Synthesis of SWNT-Pt(IV) Conjugates, SWNT-Pt(IV). c,c,t-[Pt(NH₃)₂Cl₂(OEt)-(O₂CCH₂CH₂CO₂H)] was bound to the surface of the SWNT-NH₂ through peptide linkages formed between the free carboxylate on the platinum complex and the amino functionalities of the nanotubes. In a typical coupling reaction, a 1.0 mM aqueous solution of N-hydroxysuccinimide (NHS) was added to an equal volume of an aqueous 1.0 mM solution of 1-ethyl-3[3-dimethylaminopropyl]carbodiimide hydrochloride (EDC) and the resulting solution was allowed to stand at room temperature for 10 min. To this solution was added 0.8 molar equivalents of c,c,t-[Pt(NH₃)₂Cl₂(OEt)(O₂CCH₂CH₂CO₂H)] in ddH₂O. After 10 min, a solution of SWNT-NH₂ was added such that the mole ratio of amine-to-Pt was 0.5. The solution was heated to 50° C. for 2 h and then agitated overnight at room temperature. The solution was dialyzed against deionized water in a 3500 MW cutoff dialysis cassette for 10 h, changing the water at 5 h. The bound platinum concentration was subsequently determined by AAS.

Synthesis of SWNT-Pt(IV)-F1 Conjugates. SWNT-Pt(IV) conjugates were prepared as described above except with an amine-to-Pt ratio of 2:1. These constructs were then allowed to react with a 0.4 mM aqueous solution of 6-carboxy-2′,7′-dicchlorofluorescein-3′,6′-diacetatesuccinimidyl ester for 12 h. The resulting solution was dialyzed against water using a 3500 MW cutoff dialysis cassette for 6 h and the platinum concentration was determined by AAS. The resulting solution had an approximately 1:1 ratio of platinum-to-fluorophore.

### Example 2

### Electrochemical Studies of Compounds 1 and 2

Each platinum complex was dissolved to a final concentration of 2.0 mM in 0.1 M aqueous KCl buffered with phosphate to either pH 6.0 or 7.4. Cyclic voltammetric (CV) measurements were performed with a model 263 EG&G Princeton Applied Research electrochemical analyzer at varying scan rates of 20-100 mV s⁻¹. The solvent was degassed by several freeze-pump-thaw cycles and measurements were taken under an atmosphere of argon. The working electrode was made of glassy carbon, the reference electrode was Ag/AgCl, and the counter electrode was a platinum wire. Reported potentials are extrapolated to 0.0 mV s⁻¹ scan rates to account for the irreversible behavior of the reduction processes.⁷

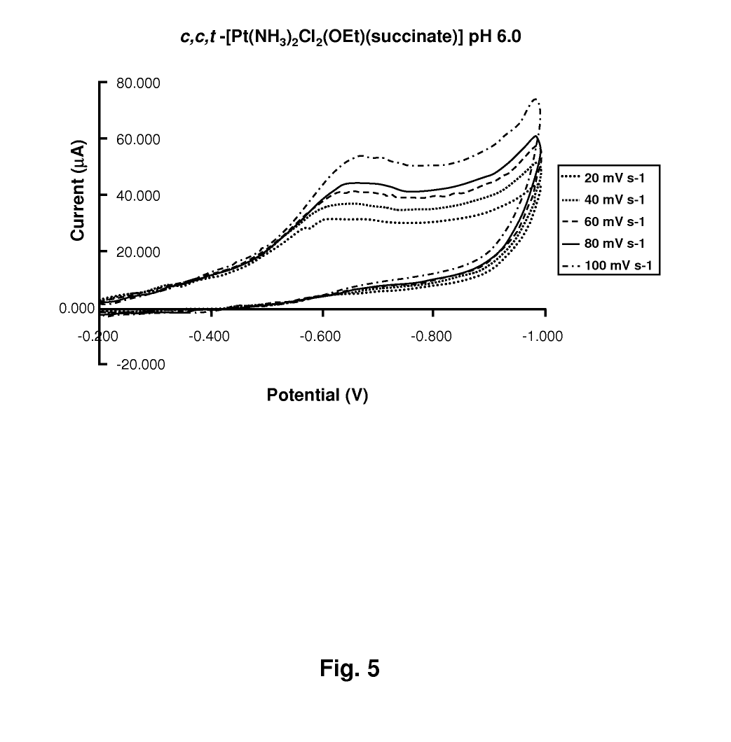

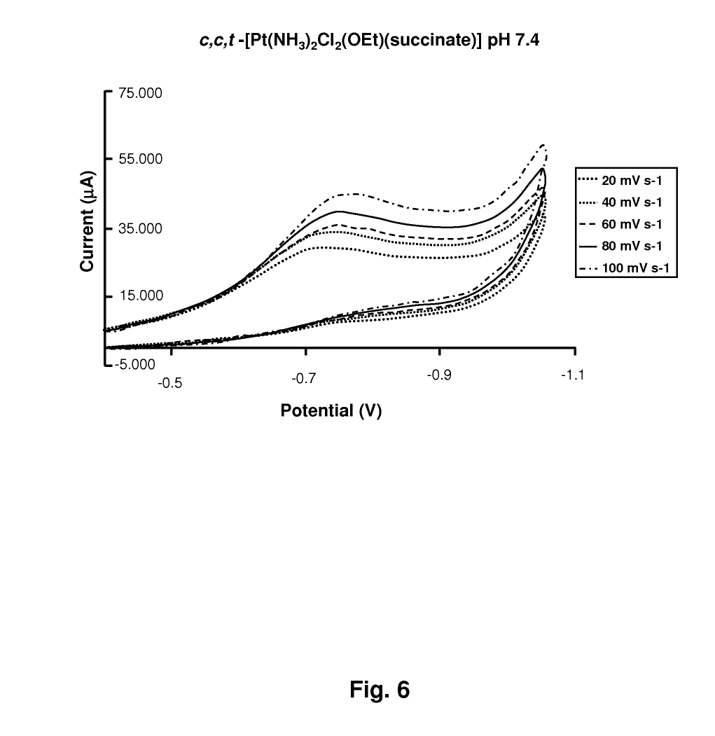

Electrochemical studies of compounds 1 and 2 showed the expected irreversible reduction maxima in their cyclic voltammograms corresponding to loss of the axial ligands. At pH 7.4 the reduction potentials extrapolated to 0.0 mV s⁻¹ scan rate for 1 and 2 are −0.604 and −0.724 V, respectively. At pH 6.0, a value similar to that reported for endosomes and lysosomes,^{6a, 6b} there is a positive shift in the reduction potentials of both complexes by greater than 100 mV, the respective reduction peaks occurring at −0.499 and −0.623 V. The more facile reduction reflects protonation of the axial ligands as leaving groups. This property will significantly affect the activity of these complexes in the low pH environment of many solid tumors and inside the endosomes. Exemplary results for the succinate-linked prodrug are illustrated in FIG. 5 (pH 6.0) and FIG. 6 (pH 7.4).

### Example 3

### Cell Cultures Showing Cytotoxicity of Compounds as Taken up by Cells

Cell Culture. Cells from the human testicular cancer line NTera-2 were incubated at 37° C. in 5% CO₂ and grown in DMEM (Dulbecco's Modified Eagle's Medium) medium supplemented with 10% fetal bovine serum and 1% penicillin/streptomycin. Cells were passaged every 3 to 4 days and reseeded from frozen stocks after reaching passage number 20.

Cell Fixing Solution. Paraformaldehyde (4.0 g) and NaOH (0.4 g) were dissolved in 100 mL of distilled water. A 1.68 g portion of NaH₂PO₄ was added and the pH was adjusted with NaOH and HPO₄ to be between 7.5 and 8.0. Sucrose (4.0 g) was added and the resulting solution was stored at 4° C. until use.

Cell Extract Preparation and Analysis. NTera-2 cells were grown to _{>}90% confluence in 175 cm² flasks. These cells were treated separately with 1 μM concentrations of cisplatin, c,c,t-[Pt(NH₃)₂Cl₂(OEt)(O₂CCH₂CH₂CO₂H)], and SWNT-Pt(IV) and subsequently incubated for three h at 37° C. The cells were washed thrice with PBS and released by trypsinization into PBS. The cell solutions were centrifuged at 800×g for 10 min and the cell pellets resuspended in 100 μL of ice-cold lysis buffer (1.0 mM DTT (dithiothreitol), 1.0 mM PMSF (phenylmethanesulphonylfluoride), 10 mM KCl, 10 mM MgCl₂, pH 7.5) for 15 min. The cells were centrifuged again and the pellets resuspended in 40 μL of ice-cold lysis buffer. The cell membranes were lysed by 10 strokes of a 28 ga. syringe. The resulting suspension was centrifuged at 11,000×g for 20 min and the supernatant was collected as the cytosolic fraction of the cells. The pellet was resuspended in 40 μL of extraction buffer (1.0 mM DTT, 1.0 mM PMSF, 1.5 mM MgCl₂, 0.2 M EDTA, 0.42 M NaCl, 25% glycerol, pH 7.9) and lysed with 10 strokes of a 28 ga. syringe. The lysate was shaken at 1000 rpm for 45 min at 40° C. and then centrifuged at 20,000×g for 10 min at 4° C. The supernatant was collected as the nuclear fraction. The platinum concentration of all fractions was determined by AAS. The protein concentration for each fraction was determined by the bicinchoninic acid (BCA) assay and the total platinum concentration was expressed as nanograms of platinum per microgram of protein (Table 1).

TABLE 1
Platinum Concentration of Cell Extracts Expressed as Pt(ng)/protein(mg).

| Platinum Source | Cytosolic | Nuclear |
|---|---|---|
| cis-[Pt(NH₃)₂Cl₂] | 11 | 14 |
| c,c,t-[Pt(NH₃)₂Cl₂(OEt)(O₂CCH₂CH₂CO₂H)] | 3 | 6 |
| SWNT-Pt(IV) | 23 | 36 |

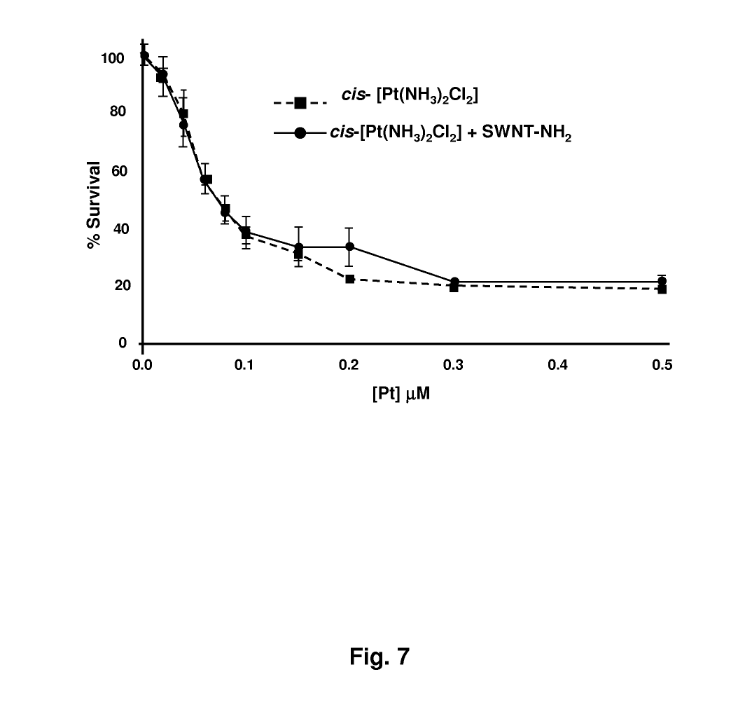

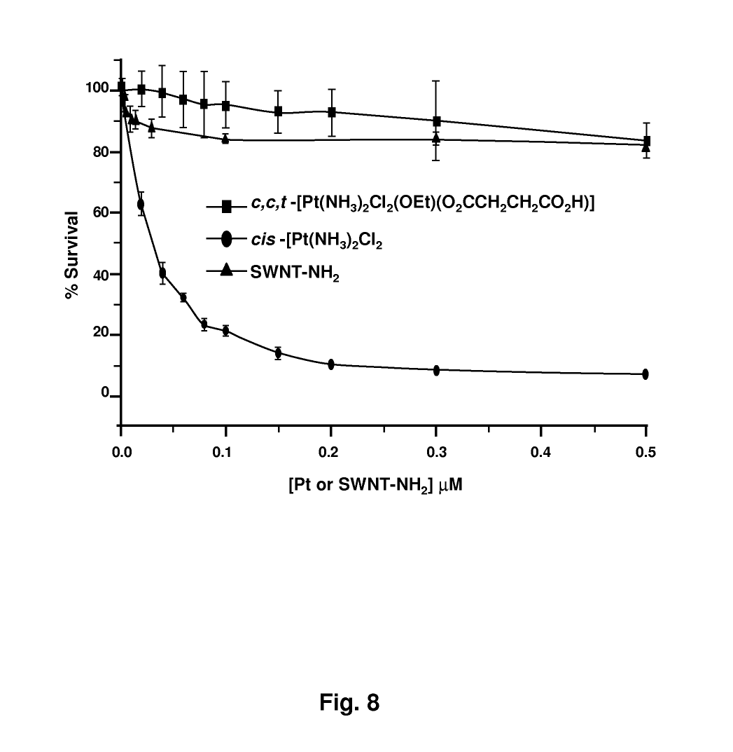

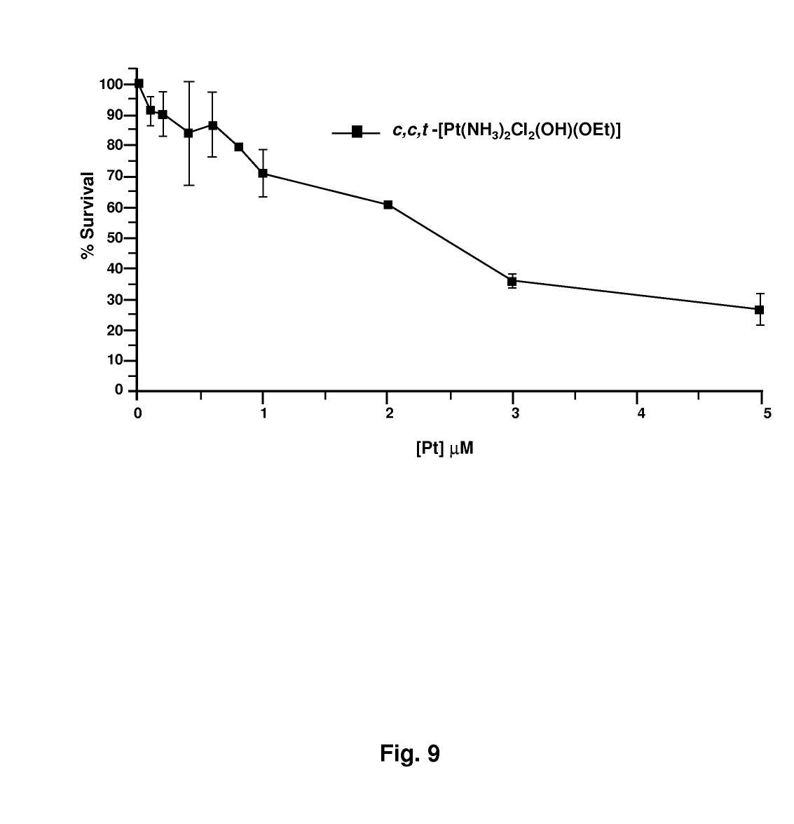

Cell Proliferation Assay. Ntera-2cells were seeded into 96 well plates at a confluence of 1000 cells per well and incubated for 24 h. The cells were then treated with either cisplatin, compound 1, compound 2, or SWNT-Pt(IV) and incubated for 96 h. The cells were then treated with 20 μL of 3-(4,5-dimethylthiazol-2-yl)-2,5-diphenyltetrazolium bromide (MTT) (5 mg/mL in PBS) and incubated for 5 h. The medium was removed, the cells were lysed with 100 μL of DMSO, and the absorbance of the purple formazan was recorded at 550 nm. The results are presented in FIG. 2, FIG. 7, FIG. 8, and FIG. 9. FIG. 2 shows that the compound containing platinum IV did not kill the cancer cells tested unless conjugated to the nanoparticles. FIG. 7 shows that the SWNTs do not affect the cytotoxicity of the cis-[Pt(NH₃)₂Cl₂] (cisplatin). FIG. 8 shows that the cis-[Pt(NH₃)₂Cl₂] (cisplatin, circles) is significantly more toxic to the cells tested than either the nanoparticles themselves (SWNT, triangles) or the prodrug compound 2. FIG. 9 shows that the compound 1, c,c,t-[Pt(NH₃)₂Cl₂(OH)(OEt)] is only toxic to cells in micromolar concentrations.

Fluorescence Microscopy Experiments on Testicular Cancer Cells. NTera-2 cells were plated on 1 cm microscope coverslips at a confluence of 30000 cells per slip and incubated overnight. The growth medium was then exchanged for DMEM containing the SWNT-Pt(IV)-F1 conjugates at a final fluorophore concentration of 1.0 μM, and the cells were incubated for 1.5 h and 3.0 h at 37° C. The DMEM was then removed and the cells were incubated with a fixing solution for 10 min at room temperature followed by 3 washes with phosphate buffered saline (PBS). A PBS solution of Hoechst (H33258) nuclear stain (250 μg/L) was administered to the cells for 10 min at room temperature. The cells were then washed with Millipore water and mounted on microscope slides for imaging. Images were collected at 500 msec for the DAPI (4′,6-diamidino-2-phenylindole) channel and 270 msec for the FITC (Fluorescein isothiocyanate) channel. The resultant fluorescent images showed that the green dye label of the SWNTs was visible in the cytoplasm, and the nuclei stained blue. The cellular distribution of a similar type of SWNT after 24 h of incubation was previously reported.⁸

In summary, fluorescence images of NTera-2 cells after 3 h incubation with SWNT-Pt(IV)-F1 showed that the nuclei stained blue and a green color revealed internalization of the SWNTs. Fluorescence image of NTera-2 control cells with no SWNT-Pt(IV)-F1 showed no green staining.

### REFERENCES

1. (1)(a) Galanski, M.; Jakupec, M. A.; Keppler, B. K., Curr. Med. Chem. 2005, 12, 2075-2094.(b) Wong, E.; Giandomenico, C. M., Chem. Rev. 1999, 99, 2451-2466.
2. (2) Wang, D.; Lippard, S. J., Nat. Rev. Drug Discovery 2005, 4, 307-320.
3. (3)Xu, P.; VanKirk, E. A.; Murdoch, W. J.; Zhan, Y.; Isaak, D. D.; Radosz, M.; Shen, Y., Biomacromolecules 2006, 7, 829-835.
4. (4)Barnes, K. R.; Kutikov, A.; Lippard, S. J., Chem. Biol. 2004, 11, 557-564.
5. (5)(a) Bottini, M.; Cerignoli, F.; Dawson, M. I.; Magrini, A.; Rosato, N.; Mustelin, T., Biomacromolecules 2006, 7, 2259-2263.(b) Gao, L.; Ni, L.; Wang, T.; Qin, Y.; Guo, Z.; Yang, D.; Yan, X., ChemBioChem 2006, 7, 239-242.(c) Kam, N. W. S.; Dai, H., J. Am. Chem. Soc. 2005, 127, 6021-6026.(d) Liu, Z.; Cai, W.; He, L.; Nakayama, N.; Chen, K.; Sun, X.; Chen, X.; Dai, H., Nat Nano 2007, 2, 47-52.(e) Nadine Wong Shi Kam, Z. L. H. D., Angew. Chem., Int. Ed. 2006, 45, 577-581.(f) Nakayama-Ratchford, N.; Bangsaruntip, S.; Sun, X.; Welsher, K.; Dai, H., J. Am. Chem. Soc. 2007, 129, 2448-2449.(g) Shi Kam, N. W.; O'Connell, M.; Wisdom, J. A.; Dai, H., Proc. Natl. Acad. Sci. U.S.A. 2005, 102, 11600-11605.(h) ShiKam, N. W.; Jessop, T. C.; Wender, P. A.; Dai, H., J. Am. Chem. Soc. 2004, 126, 6850-6851.
6. (6)(a) Arunachalam, B.; Phan, U. T.; Geuze, H. J.; Cresswell, P., Proc. Natl. Acad. Sci. U.S.A. 2000, 97, 745-750.(b) Kam, N. W. S.; Dai, H., Phys. Status Solidi B 2006, 243, 3561-3566.(c) Kam, N. W. S.; Liu, Z.; Dai, H., J. Am. Chem. Soc. 2005, 127, 12492-12493.(d) Zhuang Liu, M. W. M. H. H. D., Angew. Chem., Int. Ed. 2007, 46, 2023-2027.
7. (7)(a) Giandomenico, C. M.; Abrams, M. J.; Murrer, B. A.; Vollano, J. F.; Rheinheimer, M. I.; Wyer, S. B.; Bossard, G. E.; Higgins, J. D., Inorg. Chem. 1995, 34, 1015-1021.(b) Lee, Y.-A.; Ho Yoo, K.; Jung, O.-S., Inorg. Chem. Commun. 2003, 6, 249-251.(c) Lee, Y.-A.; Jung, O.-S., Bull. Chem. Soc. Jpn. 2002, 75, 1533-1537.(d) Young-A. Lee, O.-S. J., Angew. Chem., Int. Ed. 2001, 40, 3868-3870.
8. (8)Woodroofe, C. C.; Won, A. C.; Lippard, S. J., Inorg. Chem. 2005, 44, 3112-3120.

### Conclusion

The above specific description is meant to exemplify and illustrate the invention and should not be seen as limiting the scope of the invention, which is defined by the literal and equivalent scope of the appended claims. Any patents or publications mentioned in this specification are indicative of levels of those skilled in the art to which the patent or publication pertains as of its date and are intended to convey details of the invention which may not be explicitly set out but which would be understood by workers in the field. Such patents or publications are hereby incorporated by reference to the same extent as if each was specifically and individually incorporated by reference, as needed for the purpose of describing and enabling the method or material to which is referred.

1. A nanoparticle complex for delivery of a metal containing prodrug to a cell where the metal containing prodrug is converted to an active drug, comprising:
(a) a hydrophobic nanoparticle having an extended aromatic structure;
(b) an organic amphiphilic molecule comprising (i) a hydrophilic polymer and (ii) a hydrophobic polymer noncovalently bound to the nanoparticle; and
(c) a metal containing prodrug attached to the surface of the nanoparticle through a cleavable linkage to the hydrophilic polymer, said metal containing prodrug being activated within an endosome through reduction of the prodrug to an active drug and cleavage of the cleavable linker.

2. The nanoparticle complex of claim 1 wherein the nanoparticle is a carbon nanotube

3. The nanoparticle complex of claim 2 wherein the carbon nanotube is an SWNT.

4. The nanoparticle of claim 3 wherein the SWNT has an average length of about 50-500 nm.

5. The nanoparticle complex of claim 1 wherein the hydrophilic polymer comprises PEG and the PEG is from about 10 to 500 polyethylene oxide (PEO) units.

6. The nanoparticle complex of claim 5 wherein the PEG is amine-linked to the metal containing prodrug.

7. The nanoparticle complex of claim 1 where the hydrophilic polymer comprises PEG having two to seven branches.

8. The nanoparticle complex of claim 7 wherein the hydrophilic polymer has four branches.

9. The nanoparticle complex of claim 1 comprising at least two drug molecules linked to branches of branched PEG.

10. The nanoparticle complex of claim 1 wherein the hydrophilic polymer is dextran.

11. The nanoparticle complex of claim 1 wherein the hydrophilic polymer is further linked to a targeting agent.

12. The nanoparticle complex of claim 1 wherein the hydrophilic polymer is comprised in an organic amphiphilic molecule.

13. The nanoparticle of claim 1 wherein the organic amphiphilic molecule comprises a polar lipid adsorbed on the nanoparticle through supramolecular hydrophobic bonding.

14. The nanoparticle of claim 1 wherein the polar lipid is a phospholipid.

15. The nanoparticle complex of claim 1 wherein the cleavable linkage is a linkage which is one of hydrazone, ester or disulfide.

16. A preparation of the nanoparticle complex of claim 1 in an aqueous suspension.

17. A preparation of the nanoparticle complex of claim 1 in unit dosage form.

18. The nanoparticle complex of claim 1 wherein the hydrophilic polymer is branched.

19. (canceled)

20. The nanoparticle complex of claim 1 wherein the metal containing prodrug is an anticancer drug selected from the group consisting of gallium, platinum, palladium, lanthanide series, ruthenium, osmium, copper, rhodium, iridium, titanium and gold.

21. The nanoparticle complex of claim 20 wherein the metal containing prodrug is a platinum containing drug which is Pt (IV) prior to delivery and Pt (II) after delivery.

22. The nanoparticle complex of claim 20 where the metal containing prodrug comprises an axial ligand comprising an alkoxide group.

23. A method for preparing a nanoparticle complex for delivery of a metal containing prodrug of a reduced active agent inside a cell, comprising the steps of:
(a) obtaining a nanoparticle, which has an extended aromatic surface, in dispersed form;
(b) attaching a hydrophilic polymer to the nanoparticle;
(c) attaching noncovalently the hydrophilic polymer to the nanoparticle, and linking the prodrug to the hydrophilic polymer; and
(d) forming a stable aqueous suspension of the complex.

24. A method for delivering delivery of a metal containing prodrug for conversion to an active agent inside a cell, comprising the step of administering the metal containing prodrug in a complex comprising:
(a) a nanoparticle having a graphitic surface;
(b) an organic amphiphilic molecule comprising a hydrophilic polymer noncovalently bound to the nanoparticle through a hydrophobic polymer; and
(c) a metal containing prodrug linked to the hydrophilic polymer through a cleavable linkage, there being between ten and five hundred hydrophiic polymers and prodrugs per nanoparticle, said method comprising the step of:
(d) contacting the cell with the complex for a time sufficient to allow internalization of the complex.

25. The method of claim 24 where the hydrophilic polymer further comprises a targeting agent for delivering the active agent to a cell type providing a target for the targeting agent.

26. The method of claim 24 where the metal containing prodrug is converted to a reduced metal active agent by pH lower than 7.4.

27. The method of claim 24 comprising the step of injecting the complex in unit dosage form.

28. The method of claim 24 where the prodrug is of an active agent that is an anti-cancer drug.

29. The method of claim 24 where the prodrug is Pt (IV).

30. A nanoparticle complex for delivery of an active agent into a cell, comprising:
(a) a nanoparticle having an extended aromatic structure;
(b) an amphiphilic polymer comprising PEG for binding to the nanoparticle and solubilizing it, said amphiphilic polymer further comprising a hydrophobic polymer noncovalently bound to the nanoparticle; and
(c) a metal containing prodrug coupled to the PEG by an amide linkage there being between ten and five hundred prodrug molecular complexes per nanoparticle .

31. The nanoparticle complex of claim 25 wherein the targeting agent is an RGD peptide.

32. The nanoparticle complex of claim 25 wherein the targeting agent comprises an antibody or antibody fragment.

33. The nanoparticle complex of claim 25 wherein the metal containing prodrug comprises platinum and further comprises an axial ligand comprising an alkoxide group.
